(19) United States
(12) Patent Application Publication
(10) Pub. No.: US 2022/0061795 A1
(43) Pub. Date: Mar. 3, 2022

(54) STETHOSCOPE CHESTPIECE WITH MULTIPLE CAVITIES

(71) Applicant: 3M INNOVATIVE PROPERTIES COMPANY, St. Paul, MN (US)

(72) Inventors: Daniel J. Rogers, Grant, MN (US); William Bedingham, Woodbury, MN (US); Tyler J. Sandback, Prior Lake, MN (US)

(21) Appl. No.: 17/421,329

(22) PCT Filed: Nov. 7, 2019
(86) PCT No.: PCT/IB2019/059584

§ 371 Date: Jul. 7, 2021

Related U.S. Application Data
(60) Provisional application No. 62/795,121, filed on Jan. 22, 2019.

(51) Int. Cl. A61B 7/02; G10K 13/00

(52) U.S. Cl. CPC A61B 7/02; G10K 13/00

(57) ABSTRACT

Aspects of the present disclosure relate to a stethoscope chestpiece. The stethoscope chestpiece includes a first major cavity formed from a portion of an outer wall portion. The outer wall portion includes an outer lower edge forming an outer lower edge perimeter that establishes a bottommost plane. The stethoscope chestpiece also includes a second major cavity formed from a portion of an inner wall portion having an immobilizing element disposed thereon, the second major cavity is within the first major cavity, and a portion of the immobilizing element is spaced-apart with a portion of the outer lower edge perimeter. The first major cavity volume is at least two times a second major cavity volume.

### BACKGROUND

Complete diagnosis of a patient often requires that a physician monitor both low frequency and high frequency sounds associated with, for example, the heart. In respect to the heart, it is important that the physician alternate rapidly between the monitoring of low frequency and high frequency sounds so that the physician does not lose the impression from the previously heard heartbeat before the next beat is heard. If the process of alternating between monitoring low frequency and high frequency sounds requires considerable time, a significant number of heartbeats may unfortunately go undetected.

While tunable stethoscopes exist (stethoscopes that change the amplification or attenuation of frequencies based on applied pressure), some tunable stethoscopes can have issues attenuating higher frequency sounds (frequencies above 500 Hz).

### SUMMARY

Aspects of the present disclosure relate to a stethoscope chestpiece. The stethoscope chestpiece includes a first major cavity formed from a portion of an outer wall portion. The outer wall portion includes an outer lower edge forming an outer lower edge perimeter that establishes a bottommost plane. The stethoscope chestpiece also includes a second major cavity formed from a portion of an inner wall portion having an immobilizing element disposed thereon, the second major cavity is within the first major cavity, and a portion of the immobilizing element is spaced-apart with a portion of the outer lower edge perimeter. The first major cavity volume is at least two times a second major cavity volume.

Additional aspects of the present disclosure relate to a stethoscope chestpiece that includes an outer wall portion having an outer inside face, an outer outside face, an outer lower edge, the outer lower edge forms an outer lower edge perimeter and defines a first plane. The stethoscope chestpiece also includes an inner wall portion contained within the outer portion having an inner inside face, an inner outside face, an inner lower portion, the inner lower portion forms an inner lower portion perimeter and defined by a second plane that is positioned above the first plane towards a distal end, a portion of the inner lower portion perimeter is spaced-apart from a portion of the outer lower edge perimeter and a portion of the inner outside face is spaced-apart from the outer inside face. The outer wall portion can form a first cavity having a first volume and the inner wall portion forms a second cavity having a second volume, the first volume is at least two times the second volume. The stethoscope chestpiece also includes a stem fitting partially formed in the outer wall portion such that a portion of the outer outside face is fluidically coupled to the second cavity.

When combined with a diaphragm and other components to form an auscultation device, the auscultation device can attenuate frequencies from 500 to 1500 Hz by at least 5 decibels compared to a stethoscope chestpiece (such as a Master Cardiology by 3M) that has a first volume less than two times the second volume when less than 100 g of downward force is applied.

### BRIEF DESCRIPTION OF DRAWINGS

The disclosure may be more completely understood in consideration of the following detailed description of various embodiments of the disclosure in connection with the accompanying drawings.

FIG. 1 illustrates a stethoscope, according to various aspects of the present disclosure.

FIGS. 2A-2H illustrates different views of a stethoscope chestpiece useful in the stethoscope, according to various aspects of the present disclosure.

FIG. 3 illustrates a cross-sectional view of the stethoscope chestpiece of FIGS. 2A-2H, according to various aspects of the present disclosure.

FIG. 4 illustrates a cross-sectional view of the stethoscope chestpiece of FIGS. 2A-2H and FIG. 3, according to various aspects of the present disclosure.

FIG. 5 illustrates a cross-sectional view of a stethoscope chestpiece useful in a stethoscope, according to various aspects of the present disclosure.

FIG. 6A illustrates a perspective view of a stethoscope chestpiece useful in a stethoscope, according to various aspects of the present disclosure.

FIG. 6B illustrates a cross-sectional view of the stethoscope chestpiece of FIG. 6A taken along lines 6-6, according to various aspects of the present disclosure.

FIG. 7 illustrates a graph of frequency responses between an example and a comparative example at light pressure.

FIG. 8 illustrates a graph of frequency responses between an example and a comparative example at firm pressure.

FIG. 9A-9B illustrates an exemplary stethoscope, according to various aspects of the present disclosure.

FIG. 10 illustrates an acoustic test system, according to various aspects of the present disclosure.

FIG. 11A-E illustrates an exemplary stethoscope, according to various aspects of the present disclosure.

FIG. 12 illustrates a graph of frequency responses between an example and a comparative example at light pressure.

FIG. 13 illustrates a graph of frequency responses between an example and a comparative example at firm pressure.

### DETAILED DESCRIPTION

As used in the instant specification and claims, “acoustical stiffness” of the diaphragm designates the mechanical stiffness of the diaphragm as influenced by the mechanical stiffness of the diaphragm material itself, the thickness of the diaphragm, the shape of the diaphragm, the diameter of the diaphragm, and the manner in which the diaphragm is attached to the stethoscope chestpiece. The phrase “plane of the diaphragm” refers to the generally planar surface of the diaphragm.

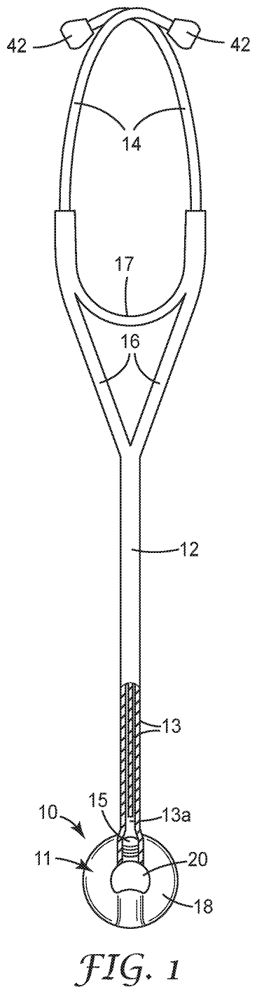

A stethoscope 100 is shown in FIG. 1. The stethoscope 100 includes a stethoscope chestpiece 10. The stethoscope chestpiece 10 comprises body member 11 formed of metallic or thermoplastic compositions. Stethoscope chestpiece 10 is attached to a conventional headset such as those commercially available under the trade designation Littmann by 3M (St. Paul, Minn.) which comprises elongated flexible tubing 12 which contains dual air passages 13 which run side-by-side for a major portion of the distance between stethoscope chestpiece 10 and ear tubes 14. In the lower end of flexible tubing 12 which attaches to stethoscope chestpiece 10, passages 13 merge into a single passage 13 a adapted to be coupled to stem fitting 15 of stethoscope chestpiece 10. The upper end of flexible tubing 12 bifurcates into coupling arms 16, each of which attaches to one of the ear tubes 14 and each of which contains one of the ear tips 42. Ear tubes 14 are secured together by tubing 17 and can form a yoke or a Y-shaped element. In at least one embodiment, the ear tips 42 can be fluidically coupled to an inner/second cavity of the stethoscope chestpiece as described further herein.

The stethoscope 100 can also include a diaphragm releasably attached to the chestpiece 10.

In at least one embodiment, a stethoscope 100 can also include electronic components, such as a microphone, a speaker, and signal processing to process the signals from the microphone and the speaker. In at least one embodiment, the microphone can be positioned to collect auscultation sounds from the diaphragm.

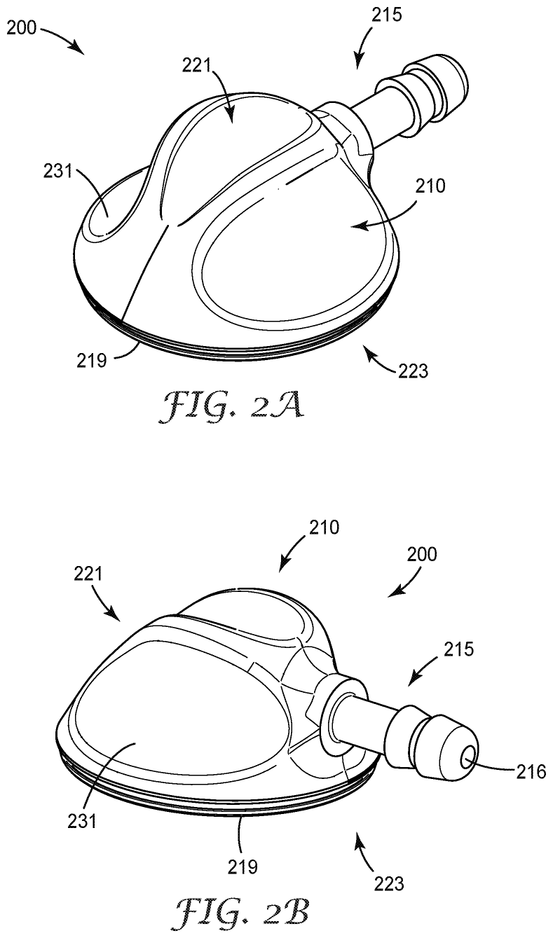

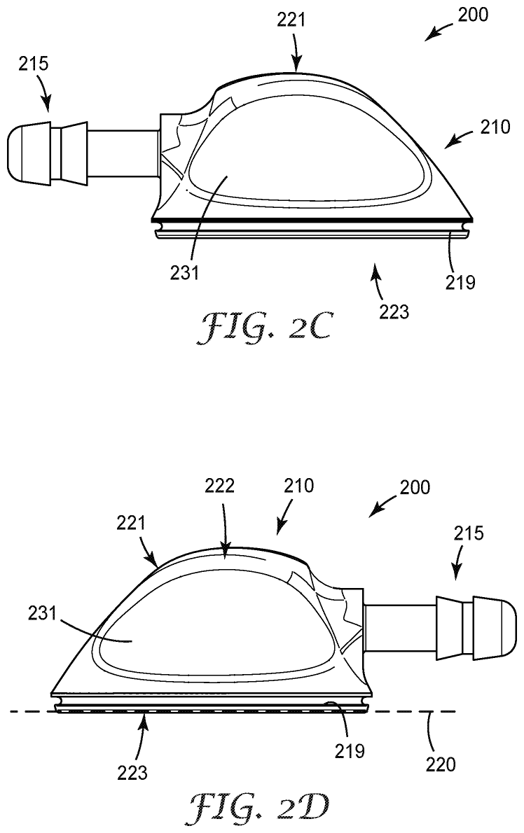

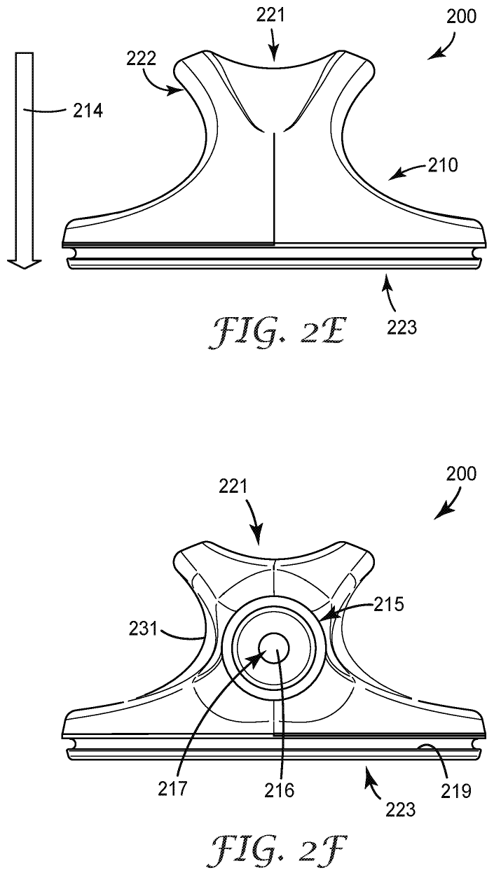

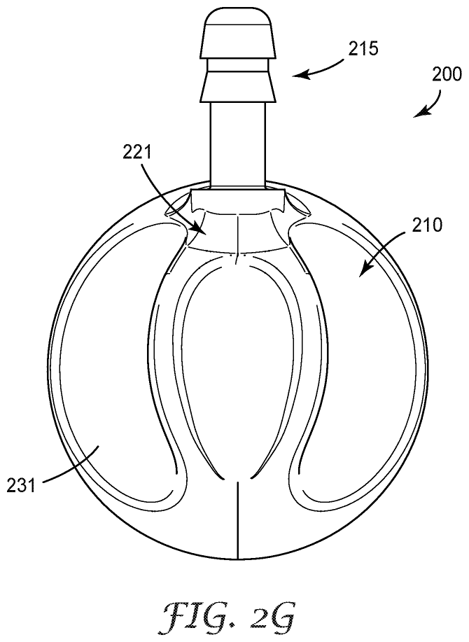

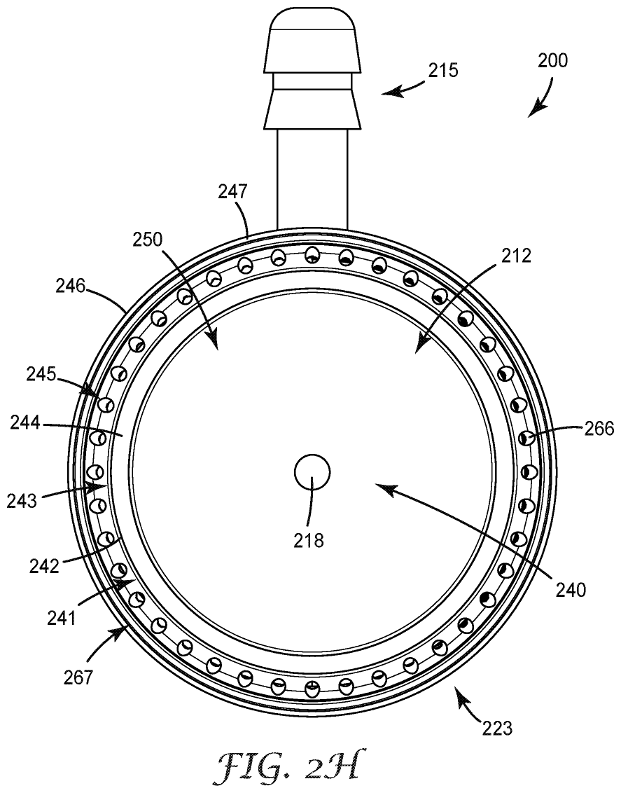

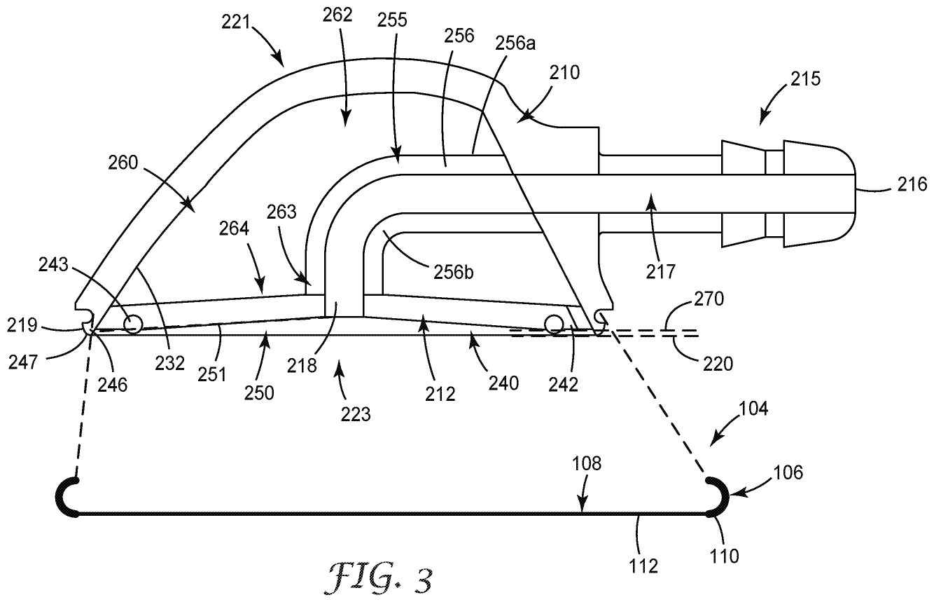

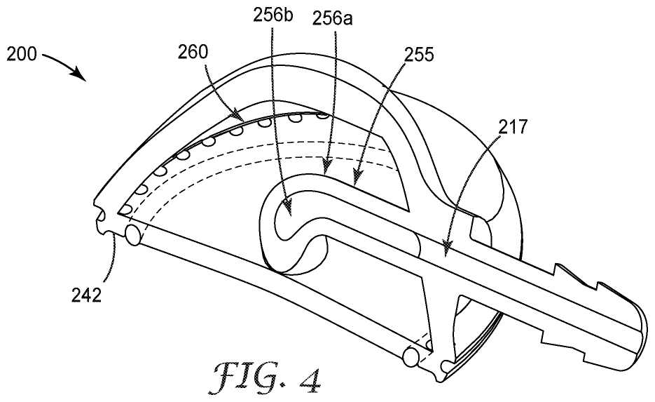

FIGS. 2A-2H illustrate different views of a stethoscope chestpiece 200. FIGS. 3 and 4 illustrate different cross-sectional views of the stethoscope chestpiece 200. In at least one embodiment, the stethoscope chestpiece 200 can be an embodiment of chestpiece 10 from FIG. 1. The stethoscope chestpiece 200 can have two major cavities wherein one major cavity has a volume of at least two, at least 3, at least 4, at least 5, at least 6, at least 7, at least 8, at least 9, at least 10 times a volume of another major cavity.

In at least one embodiment, the stethoscope chestpiece 200 is single-sided meaning that only one diaphragm can be disposed on the chestpiece 200 at a time. Examples of single-sided chestpieces 200 can include the model Master Cardiology by 3M (Saint Paul, Minn.).

In use, pressure can be applied to the chestpiece 200 in a downward 214 direction toward a patient as shown in FIG. 2E. The pressure can be capable of causing a portion of a diaphragm to contact an immobilizing element 243. An aspect of the present disclosure is two major cavities within the chestpiece 200 amplifying sounds at a frequency at least 300 Hz. For example, the sounds at frequencies above 300 Hz can be amplified by at least 5 decibels, at least 10 decibels, or at least 15 decibels such as when pressure is applied to the chestpiece.

The chestpiece 200 can be formed from one or more portions. For example, the chestpiece 200 can include an outer wall portion 210 and an inner wall portion 212. The outer wall portion 210 can form a major cavity 260 from walls of the outer wall portion 210. The outer wall portion 210 can form a cup-like or dome-shaped portion that sits over, or partially encapsulates the inner wall portion 212. The outer wall portion 210 can have an outer surface, or outer outside face 231. In at least one embodiment, the major cavity 260 can have a volume of at least 4 ml, at least 5 ml, at least 6 ml, or at least 7 ml.

In at least one embodiment, the inner wall portion 212 can be positioned at least partially within the major cavity 260. In at least one embodiment, the inner wall portion 212 can be removably or fixedly coupled to the outer wall portion 210. For example, a bridging element can couple both the outer wall portion 210 and the inner wall portion 212. In at least one embodiment, the bridging element can be a bore tube 255 which is described further herein.

The chestpiece 200 can include a distal end 221 where a user grasps the chestpiece 200. For example, a portion of the outer outside face 231 can form handles 222 that are formed at least proximate to the distal end 221. The handles 222 can be used by the user for grasping the chestpiece 200. The distal end 221 can protrude from a diaphragm end 223. The diaphragm end 223 can have a patient-facing section that is configured to hold a diaphragm. For example, part of outer outside face 231 (proximate the diaphragm end 223) can include a first lip section 219 for attaching a diaphragm thereto.

As shown in FIG. 3, a diaphragm 104 can be paired with the chestpiece 200. In preferred embodiments of the present disclosure, the diaphragm 104 is preferably single piece, meaning that there is no separate elastomeric retaining ring 106 that is separate from the disc 108. Examples of the diaphragm 104 can be found in U.S. Patent Application publication US 20180008227A1. As shown, an edge of the retaining ring 106 can contact a portion of the lip section 219. The disc 108 or ring 106 can contact the outer lower edge 246 at point 110. When downward force 214 is applied, then the non-patient facing side (inner side) of the disc 108 can contact the immobilizing element 243 at point 112 in addition to point 110. In at least one embodiment, the non-patient facing side of the diaphragm 104, when attached to the chestpiece 200, can define the major cavity 260 and/or the major cavity 240 (when a downward force 214 is applied).

The outer wall portion 210 can also have an inside surface, or inside face 232 that can form at least a portion of the major cavity 260. In at least one embodiment, the inside surface 232 can form an apex 262 in the direction of the distal end 221. The apex 262 can be considered the top most section of the inside surface 232. In at least one embodiment, a bridging element (such as a portion of the inner wall portion 212 or bore tube) can be present between the apex 262 and the inner wall portion 212.

The outer wall portion 210 can also have at least one edge, e.g., an outer lower edge 246. The outer lower edge 246 can form an outer lower edge perimeter 247 and can further define a first plane 220. The first plane 220 can be a bottommost plane of the chestpiece 200. The plane 220 defined by the outer lower edge 246 can be a boundary of the major cavity 260. In at least one embodiment, the apex 262 and/or inside face 232 can also form part of a boundary of the major cavity 260.

In at least one embodiment, the outer lower edge perimeter 247 can be defined based on the contact with a diaphragm at the point 110 the diaphragm is not immobilized. For example, as shown, the outer lower edge 246 forms a defined point where a diaphragm would contact and flex freely toward the center. If the outer lower edge 246 is substantially planar with a diaphragm (i.e., forming a rectangular or square cross section), then the outer lower edge 246 can be defined by an interior corner of the rectangular or square cross-section where the point 110 of diaphragm 104 would flex.

In at least one embodiment, volume of the major cavity 260 can be defined as the void between the outer wall portion 210 and the inner wall portion 212. In at least one embodiment, the volume of the major cavity 260 can be the total volume of a liquid placed in the outer wall portion 210 (measured from the outer lower edge 246 to the apex 262 with the volume of the major cavity 240 subtracted therefrom). For example, references to the volume of the major cavity 260 does not include the volume of the major cavity 240. While multiple volumes are possible depending on the configuration, as an example, if the outer lower edge perimeter 247 is about 135 millimeters, then the volume of the major cavity can be at least 11 milliliters.

In at least one embodiment, the outer wall portion 210 can include a stem fitting 215 disposed thereon. In at least one embodiment, a portion of the stem fitting 215 can be integrated with the outer wall portion 210. The stem fitting 215 can be releasably coupled to a portion the outer wall portion 210. For example, a portion of the stem fitting 215 can be threaded and a portion of the outer wall portion 210 can be threaded such that the portion of the stem fitting 215 can be mated with a portion of the outer wall portion 210. Another portion of the stem fitting 215 can mechanically couple to flexible tubing as described herein. As shown herein, the stem fitting 215 can have a hole 216 formed from a wall of the stem fitting 215 therein. The stem fitting 215 can also include a bore tube that connects to the inner wall portion 212.

As mentioned herein, the chestpiece 200 also includes an inner wall portion 212. The inner wall portion 212 can receive sounds from a patient by using a major cavity 240 to amplify sounds. The major cavity 240 can also be referred to as a bell. The inner wall portion 212 can be contained within a major cavity 260 formed at least partially within the outer wall portion 210. The inner wall portion 212 can form a major cavity 240. The inner wall portion 212 can have an inner inside face 264 and an inner outside face 250. The inner inside face 264 can face toward the cavity 260 and the inner outside face 250 can face toward a patient and form at least a portion of the major cavity 240. In at least one embodiment, the inner inside face 264 can have a connection point 263 for a bore tube 255 described further herein.

The major cavity 240 can be indented meaning a portion of the wall forming the major cavity 240 is higher (in the distal direction) than plane 220 (formed by the outer lower edge perimeter 247). The major cavity 240 can be bordered by the inner lower portion perimeter 242. The major cavity 240 can be conical-shaped and have a central portion that is configured to direct auscultation sounds into a bore 217 that is fluidically coupled to the stem fitting 215. The major cavity 240 can be partially within major cavity 260. An aspect of the present disclosure is that the major cavity 260 is fluidically coupled to the major cavity 240 and that a volume of the major cavity 260 is at least three times, at least four times, at least five times, at least six times, at least seven times, at least eight times, at least nine times, or at least ten times the volume of the major cavity 240 (or the ratio of the first major cavity 260 to the second major cavity 240 is at least 2:1, at least 3:1, at least 4:1, at least 5:1, at least 6:1, at least 7:1, at least 8:1, at least 9:1, at least 10:1.)

The inner wall portion 212 can have at least one inner lower portion 241. The inner lower portion 241 can form an inner lower portion perimeter 242. As shown the inner lower portion 241 of the inner wall portion 212 is proximate to the immobilizing element 243, The inner lower portion 241 can mark a boundary of the major cavity 240. In at least one embodiment, the inner lower portion 241 can be a depression or raised-portion relative to the surface of the inner wall portion 212 along the major cavity 240. For example, a groove can indicate that a bottommost portion of major cavity 240 is planar with the outer lower edge perimeter 247. The inner wall portion 212 can also include an inner lower edge 267 which forms a boundary of the inner wall portion 212. For example, the inner lower edge 267 can be the end of the inner wall portion 212.

In at least one embodiment, the inner lower portion perimeter 242 can be at least partially spaced-apart from the outer lower edge perimeter 247 of the outer wall portion 210. Partially spaced-apart means that a gap (such as an air gap) exists between the outer lower edge perimeter 247 and the inner lower portion perimeter 242 such that sound can travel from the major cavity 240 to the major cavity 260 through the gap.

The inner outside face 250 can also include an immobilizing element 243. The immobilizing element 243 can be configured to contact a portion of a diaphragm (104 as shown in FIG. 3) in response to a downward 214 force applied. For example, the immobilizing element 243 can be adapted to be contacted by a diaphragm and to substantially immobilize a diaphragm when the diaphragm is in an inner position so that a stethoscope chestpiece/head will pass low frequency sounds and attenuate high frequency sounds when the diaphragm is in an outer position and between the outer and inner positions. When the diaphragm is in the inner position the acoustical stiffness of the diaphragm can be sufficiently higher than the first acoustical stiffness so that the head will pass high frequency sounds and attenuate low frequency sounds.

In at least one embodiment, the inner outside face 250 of the inner wall portion 212 includes the immobilizing element 243 disposed thereon and contained within the major cavity 240. The immobilizing element 243 can have an immobilizing element perimeter 244 which is defined as a perimeter where a diaphragm, when downward pressure is applied to the chestpiece, that contacts the diaphragm. In at least one embodiment, the immobilizing element perimeter 244 is the topmost portion of the immobilizing element 243. The immobilizing element perimeter 244 can also be the innermost (facing toward the major cavity 240) portion of the immobilizing element 243 depending on the dimensions of the immobilizing element 243. In at least one embodiment, the immobilizing element perimeter 244 can indicate a boundary of the major cavity 240.

In at least one embodiment, at least a portion of the immobilizing element 243 can be a shelf raised relative to a plane (e.g., 251 from FIG. 3) of a major cavity 240. The immobilizing element 243 can be disposed proximate to an inner lower portion perimeter 242. In at least one embodiment, the immobilizing element 243 can include an elastomeric ring (described herein) disposed proximate or adjacent to the inner lower portion perimeter such that there is a change in frequency response when a downward 214 force is applied. The immobilizing element 243 can also include a raised portion relative to the plane (e.g., 251 in FIG. 3) of the face 250. For example, the raised portion of the immobilizing element 243 that is at least partially spaced apart from the outer inside face 232. In at least one embodiment, the raised or depressed portion (relative to the face 250) can retain the elastomeric portion. The raised portion can form a portion of the immobilizing element perimeter 244.

The immobilizing element perimeter 244 can be raised relative to a plane (e.g., 251 in FIG. 3, following contours of the cavity 240 of the inner outside face 250).

In at least one embodiment, the immobilizing element perimeter 244 can be defined by a plane 270 substantially parallel to a body surface of a patient. For example, opposing ends of the immobilizing element 243 (e.g., an inner lower edge perimeter 244) can form the plane 270. In at least one embodiment, opposing ends of the outer lower edge perimeter 247 can form a plane 220. The plane 270 can be positioned above the plane 220 in the distal direction (i.e., in the direction toward the distal end 221 from the diaphragm end 223). In at least one embodiment, the plane 270 and/or a plane formed by a hole 218 can define a boundary of the major cavity 240.

In at least one embodiment, a portion of the immobilizing element perimeter 244 is spaced-apart from a portion of the outer lower edge perimeter 247 and a portion of the inner outside face 250 is spaced-apart from the outer inside face 232.

In at least one embodiment, the inner wall portion 212 can maintain at least a partial air-gap 245 (which is shown as a plurality of holes 266 in the inner wall portion 212 ) between the immobilizing element 243 and the inner lower portion perimeter 242. The partial air-gap 245 can allow the major cavity 240 to be fluidically coupled to the major cavity 260. In at least one embodiment, the partial air-gap 245 can include a hole formed from a portion of the inner wall portion 212 therein. In at least one embodiment, the partial air-gap 245 can be a complete air-gap encompassing or surrounding the inner lower portion perimeter 242 or the immobilizing element perimeter 244 and the outer lower edge perimeter 247. In another example, a plurality of holes can be formed from the inner wall portion 212 therein, the outer wall portion 210 therein, or combination of aligning indentations on both the inner wall portion 212 and outer wall portion 210, can allow the fluidic coupling between the major cavity 240 and major cavity 260. In another example, an edge of the immobilizing element 243 can float (be unattached such that an air-gap is maintained) relative to the outer lower edge perimeter 247. In another example, immobilizing element 243 can be attached through bridging elements (not shown) relative to the outer lower edge perimeter 247 or the inner lower portion perimeter 242.

In at least one embodiment, a vibration dampening material (such as elastomeric materials) can be added between the inner wall portion 212 or immobilizing element 243 and the outer wall portion 210 (proximate an air-gap) between at least part of the inner lower portion perimeter 242 and the outer lower edge perimeter 247. Various combinations of air-gaps and materials of the aforementioned are also possible.

In at least one embodiment, the inner wall portion 212 can contact the outer wall portion 210. For example, the inner wall portion 212 can be secured to the outer wall portion 210 via a bridging element. In at least one embodiment, the inner wall portion 212 is integrally formed with the outer wall portion 210. The inner wall portion 212 can also be a separate component from the outer wall portion 210. For example, the outer wall portion 210 can be releasably attached to the inner wall portion 212. In at least one embodiment, the outer wall portion 210 can have an engagement mechanism (i.e., a seat, shelf, or lip) that is configured to mate with part of the inner wall portion 212 (e.g., proximate the inner lower edge 267).

In at least one embodiment, the major cavity 240 can be fluidically coupled to the stem fitting 215. For example, a hole 216 formed from the stem fitting 215 can lead to a hole 218 via a bore 217. Thus, the bore 217 can provide a fluidic connection between the hole 216 and hole 218. In at least one embodiment, the bore 217 can be formed from part of the outer wall portion 210, the inner wall portion 212, or combinations thereof (e.g., such that a portion of the outer outside face 231 is fluidically coupled to the major cavity 240). In at least one embodiment, part of the bore 217 can be formed from the bore tube 255. The inner wall portion 212 can also be secured to the outer wall portion 210 via the bore tube 255. The bore tube 255 can include a wall 256 having an outer face 256 a and an inner face 256 b that forms a portion of the bore 217 therein.

FIG. 4 illustrates a perspective cross-sectional view relative to the major cavity 260. In at least one embodiment, the bore tube 255 can be positioned in and extend through major cavity 260. As shown, the outer face 256a of the bore tube 255 is facing the major cavity 260 and the inner face 256b forms the bore 217.

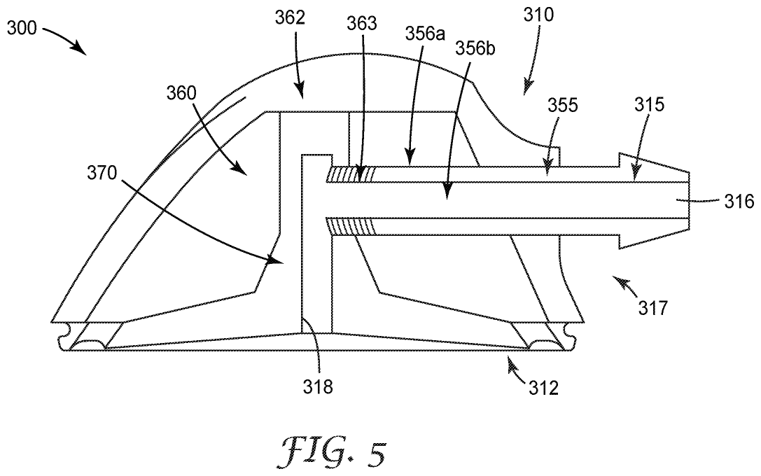

FIG. 5 illustrates an embodiment of a chestpiece 300 having an outer wall portion 310 and an inner wall portion 312 and a stem portion 315. Parts of chestpiece 300 can be similar to chestpiece 200 with similarly number components. In at least one embodiment, outer wall portion 310, inner wall portion 312, and stem fitting 315 can form three separate interconnecting sections that are assembled together.

The outer wall portion 310 can form a major cavity 360 similar to chestpiece 200. A portion of the outer wall portion 310 can form an apex 362. As pictured, the inner wall portion 312 can form a column portion 370 that can provide a portion of the bore 317 and interconnect with the stem fitting 315. The column portion 370 can couple to the outer wall portion 310 proximate to the apex 362. In at least one embodiment, a portion of the column portion 370 can be configured to mate with a portion of the outer wall portion 310, e.g., a male component pairing with a female component.

A portion of the column portion 370 can also couple with a portion of the stem fitting 315 such that the hole 318 is fluidically coupled with the hole 316 via the bore 317. Similar to stem fitting 215, the stem fitting 315 can have a bore tube 355 comprising a wall 356 that includes an outer face 356a and an inner face 356b. The stem fitting 315 can connect to the column 370 via a connection point 363 which is shown as a threaded portion that removably connects to the column 370. In at least one embodiment, the connection point 363 can be a snap fitting and the bore tube 355 (e.g., diameter) can be spaced apart from the outer wall portion 310.

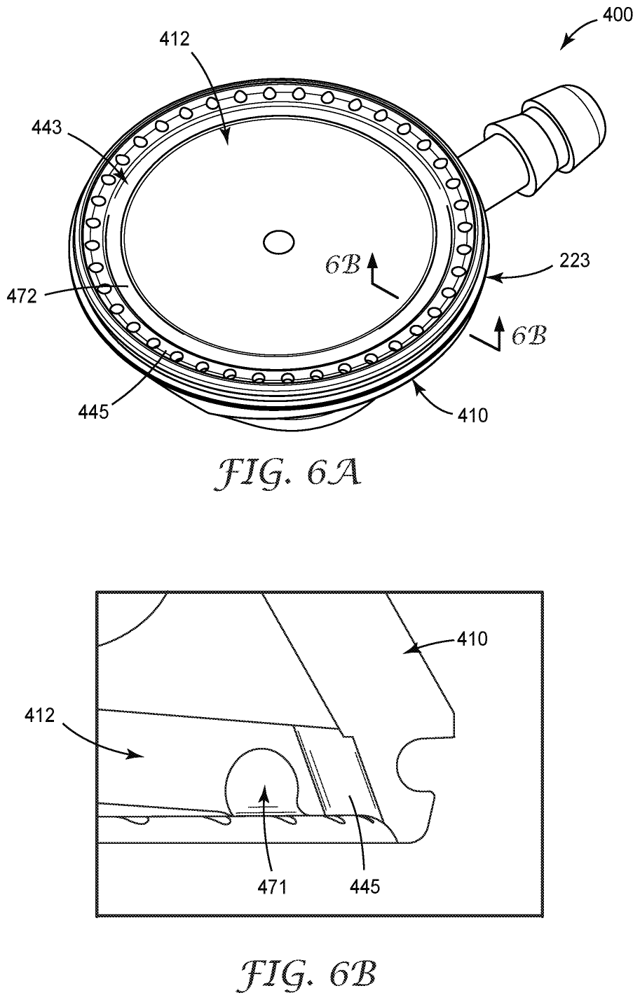

FIGS. 6A-6B illustrate an embodiment of a chestpiece 400 utilizing an O-ring 472 as an immobilizing element 443. The chestpiece 400 can be similar to chestpiece 200 with similarly number components except that the immobilizing element 443 is depicted as an elastomeric ring 472. For example, the chestpiece 400 can include an outer wall portion 410 and an inner wall portion 412. The inner wall portion can include a recess or groove 471 for seating the elastomeric ring 472. The inner wall portion 412 can also include a plurality of partial air-gaps 445 which are illustrated as holes within the inner wall portion 412.

### Examples

TABLE 1
Chestpiece Dimensions and Materials

| | | | | | First Major |
|---|---|---|---|---|---|
| | | | Outer | | Cavity |
| | | | lower edge | Immobilizing | (mL):Second |
| Stethoscope | | | perimeter | element | Major Cavity |
| Chestpiece | Material | Source | (mm) | perimeter (mm) | (mL) |
| Example 1 | VeroClear ™ | Stratasys (Eden Prairie, | 41 | 33 | 6.17:0.859 |
| | | MN) | | | (7.18:1) |
| Example 2 | VeroClear ™ | Stratasys (Eden Prairie, | 44 | 36 | 6.73:1.49 |
| | | MN) | | | (4.5:1) |
| Comparative | Stainless | Master Cardiology by | 44 | 36 | 2.97:1.49 |
| Example 1 | Steel | Littmann | | | (1.99:1) |

Examples 1, 2, and Comparative Example 1 were obtained according to Table 1.

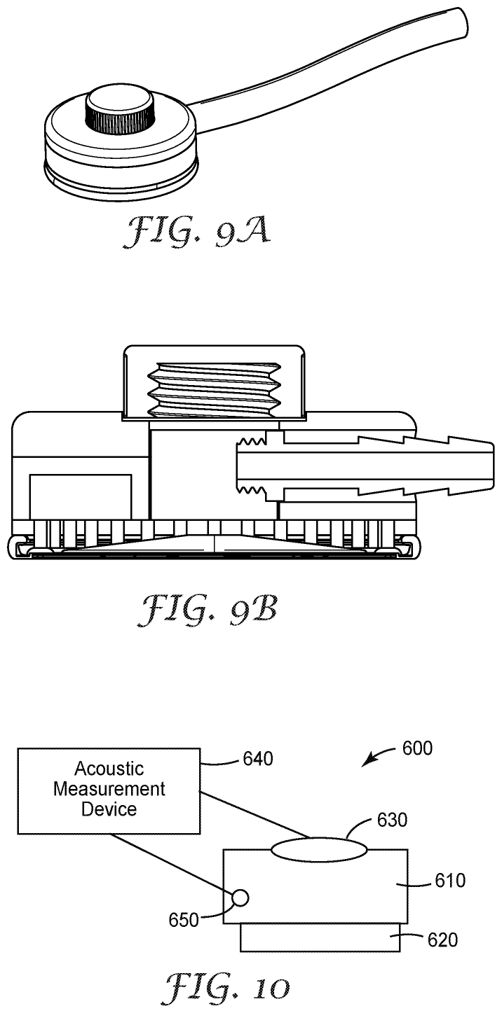

Example 1 was printed using additive manufacturing. Example 1 was formed from polymethyl methacrylate equivalent under the trade designation VeroClear which is commercially available from Stratasys (Eden Prairie, Minn.). The stethoscope chestpiece is shown on FIGS. 9A-9B. A shelf was formed similar to the Comparative Example 1. The diaphragm used was obtained from a Cardiology IV by 3M (St. Paul, Minn.).

Example 2 was printed using additive manufacturing. The stethoscope chestpiece is shown on FIGS. 11A-11E. A shelf was formed which approximates the dimensions of the Comparative Example 1. The diaphragm used was identical to that of Comparative Example 1. The differences between Example 2 and Comparative Example 1 include the cavity size, material, and the grip shape.

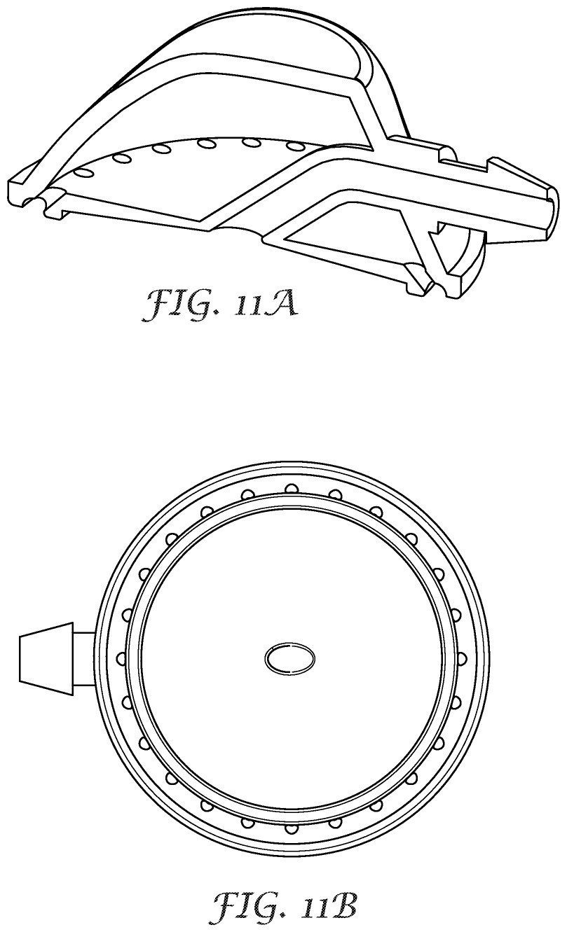

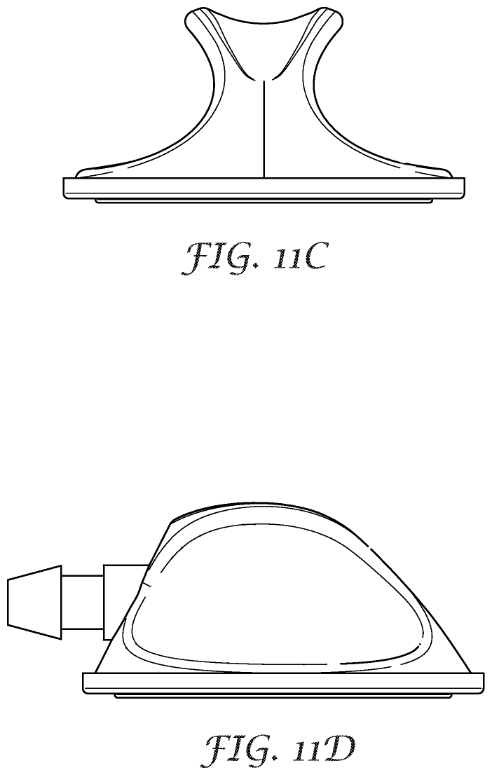

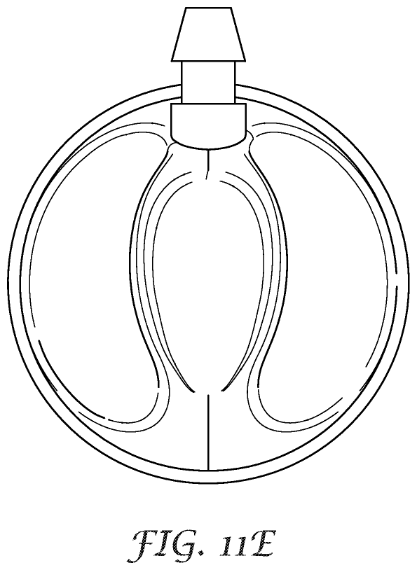

A variety of test methods can be used to test the performance of a stethoscope, e.g., an air-coupling test method. FIG. 10 illustrates a schematic view of an acoustic test system 600 to test frequency response of a stethoscope. The acoustic test system 600 can include an acoustic medium 610, an acoustic source 620, a stethoscope 630, and an acoustic measurement device 640. The acoustic medium 610 can provide a cavity coupling between the acoustic source 620 and the stethoscope 630. The acoustic medium 610 can comprise one or more coupling medium, for example, such as air, liquid, gel, foam, or the like. The acoustic medium 610 can have the shape of, for example, cylinder, cube, or the like. In some cases, the acoustic medium 610 can be sealed. The acoustic medium 610 can also provide support to the placement of the stethoscope 630. The sensor of the stethoscope 630 typically faces the cavity of the acoustic medium 610 to detect sound signals generated by the acoustic source 620. The acoustic source 620 can be, for example, a voice coil, a loudspeaker, or the like. The acoustic measurement device 640 is capable of detecting the acoustic signals. The acoustic measurement device 640 can be, for example, a Brüel & Kjær PULSE Analyzer, or a National Instrument acoustic testing system. In some implementations, a microphone 650 can be optionally included and placed inside the cavity of the acoustic medium 610 to provide reference signals. In such implementations, the frequency response of the stethoscope 630 can be indicated as the ratio of the output signals of the stethoscope verse the reference signals generated from the microphone 650 at each frequency band. In an exemplary embodiment, the acoustic test system 600 for measuring the frequency response of the Examples above includes an acoustic medium providing air cavity, a loudspeaker as the acoustic source, and a Brüel & Kjær PULSE Analyzer as the acoustic measurement device.

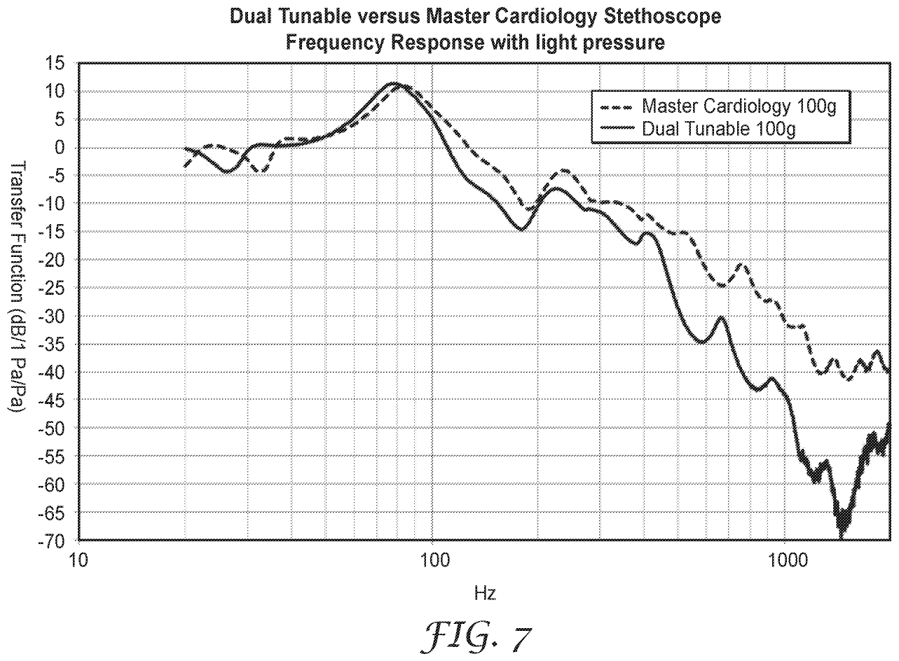

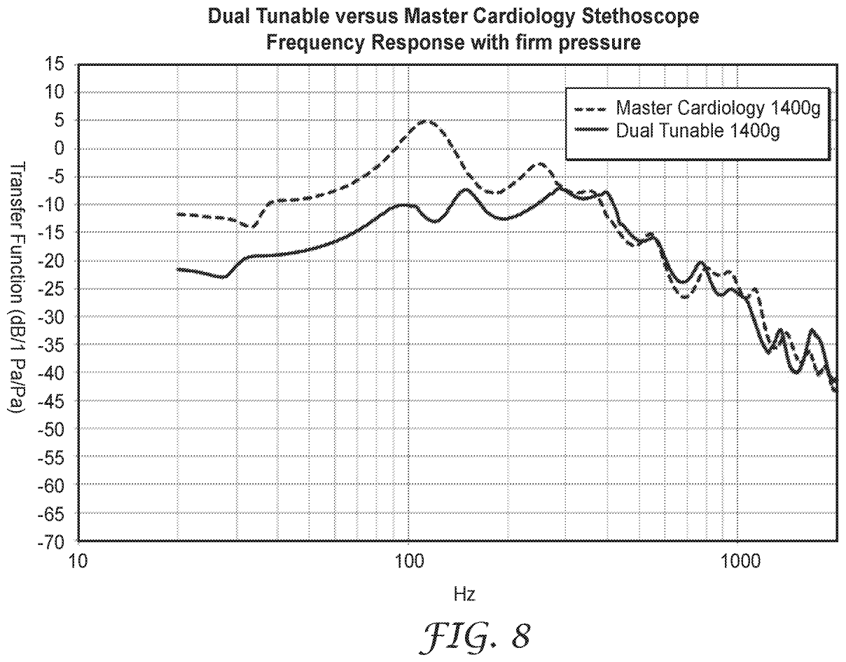

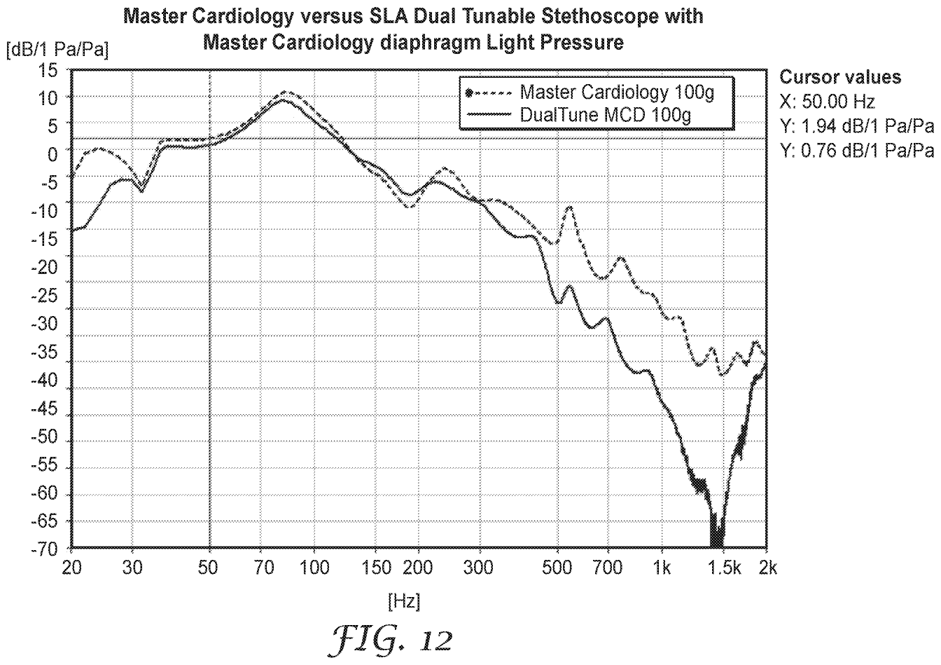

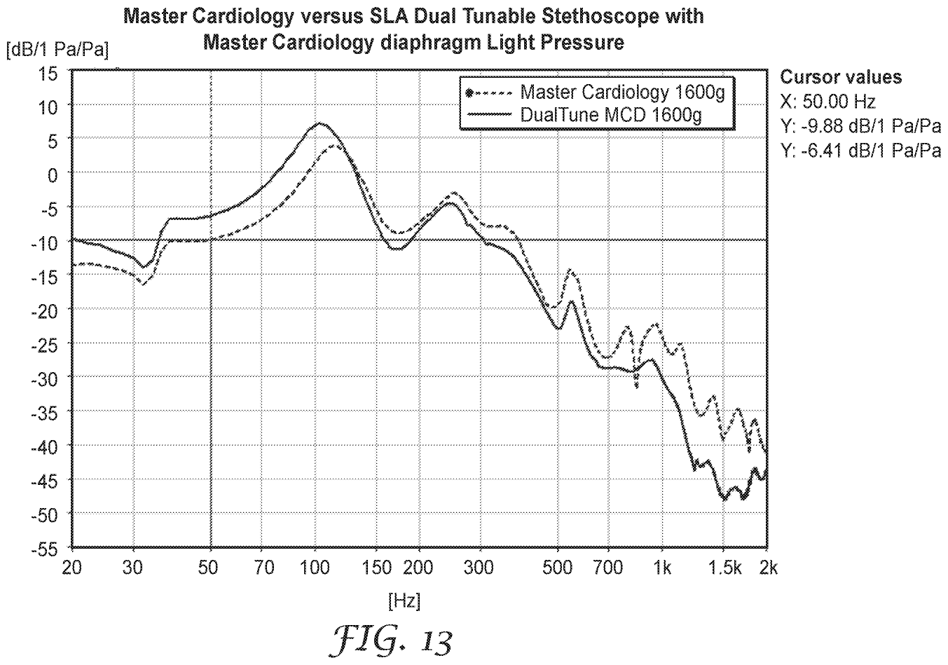

Each chestpiece was placed on the acoustic test system. The sounds were amplified by each chestpiece and the decibel level recorded. The data from both Example 1 and Comparative Example 1 is shown on FIGS. 7 and 8. The data from both Example 2 and Comparative Example 1 is shown on FIGS. 12 and 13.

With respect to the Examples, a bell of the stethoscope (e.g., formed by a conical major cavity) can be designed to focus on a bass response below 200 Hz and reduce high frequency sounds (above 500 Hz). Thus, reduction of high frequency sounds can be an objective.

With respect to Example 1, as shown, when 1400 g of downward force (e.g., firm pressure) was applied to the chestpiece, the frequency response of at least one frequency under 300 Hz (e.g., 100 Hz) is attenuated at least 5 decibels below that of Comparative Example 1. While the frequencies over 500 Hz are substantially equivalent to the Comparative Example 1. When 100 g downward force or below (e.g., light pressure) is applied, then the frequency response of at least one frequency above 400 Hz (e.g., 500 Hz) is more than 5 decibels below that of Comparative Example 1. Thus, reducing sound intensity at higher frequencies. The frequency response of frequencies below 80 Hz is substantially equivalent to the Comparative Example 1.

With respect to Example 2, as shown, when 100 g downward force or below (e.g., light pressure) is applied, then the frequency response of at least one frequency above 400 Hz (e.g., 500 Hz) is at least 5 decibels below that of Comparative Example 1. When 1600 g downward force is applied, then the frequency response below 100 Hz is substantially similar to that of Comparative Example 1.

### List of Illustrative Embodiments

1. A stethoscope chestpiece, comprising:

a first major cavity formed from a portion n outer wall portion, the outer wall portion comprises
an outer lower edge forming an outer lower edge perimeter that establishes a bottommost plane,

a second major cavity formed from a portion of an inner wall portion having an immobilizing element disposed thereon, the second major cavity is within the first major cavity, a portion of the immobilizing element is spaced-apart with a portion of the outer lower edge perimeter;

wherein the first major cavity volume is at least three times a second major cavity volume.
2. The stethoscope chestpiece of embodiment 1, wherein the immobilizing element is adapted to be contacted by a diaphragm and to substantially immobilize the diaphragm when the diaphragm contacts the immobilizing element so that the stethoscope chestpiece will pass low frequency sounds and attenuate high frequency sounds when the diaphragm is not contacting the immobilizing element, and, when the diaphragm is contacting the diaphragm, the acoustical stiffness of the diaphragm will be sufficiently higher than the first acoustical stiffness so that the head will pass high frequency sounds and attenuate low frequency sounds.
3. The stethoscope chestpiece of embodiment 1 or 2, further comprising: a bore tube positioned in the first major cavity, wherein a portion of the bore tube extends through the outer wall portion and is fluidically coupled with the first major cavity,
4. The stethoscope chestpiece of any of embodiments 1 to 3, wherein the outer wall portion comprises a distal end and a handle is formed from the outer wall portion proximate to the distal end.
5. The stethoscope chestpiece of any of embodiments 1 to 4, wherein the stethoscope chestpiece is single-sided.
6. The stethoscope chestpiece of any of embodiments 1 to 5, wherein the first major cavity volume is defined by empty space between the outer wall portion and the inner wall portion.
7. The stethoscope chestpiece of any of embodiments 1 to 6, wherein the immobilizing element is raised relative to a surface of the second major cavity.
8. The stethoscope chestpiece of any of embodiments 1 to 7, wherein the immobilizing element is an elastomeric ring.
9. The stethoscope chestpiece of any of embodiments 1 to 8, wherein attenuation of a frequency of at least 500 Hz is at least 5 decibels when downward force of less than 100 g is applied compared to a second stethoscope chestpiece.
9a. The stethoscope chestpiece of embodiment 9, wherein the second stethoscope chestpiece is under the trade designation Master Cardiology by 3M.
10. A stethoscope chestpiece, comprising:

an outer wall portion having an outer inside face, an outer outside face, an outer lower edge, the outer lower edge forms an outer lower edge perimeter and defines a first plane;

an inner wall portion contained within the outer portion having an inner inside face, an inner outside face, an inner lower portion, the inner lower portion forms an inner lower portion perimeter and defined by a second plane that is positioned above the first plane towards a distal end, a portion of the inner lower portion perimeter is spaced-apart from a portion of the outer lower edge perimeter and a portion of the inner outside face is spaced-apart from the outer inside face;

wherein the outer wall portion forms a first cavity having a first volume and the inner wall portion forms a second cavity having a second volume, the first volume is at least 3 times the second volume; and

a stem fitting partially formed in the outer wall portion such that a portion of the outer outside face is fluidically coupled to the second cavity.
11. The stethoscope chestpiece of embodiment 10, further comprising an immobilizing element disposed proximate to the inner lower portion perimeter.
12. The stethoscope chestpiece of embodiment 10 or 11, wherein the outer outside face includes a first lip section for attaching a diaphragm thereto.
13. The stethoscope chestpiece of embodiment 12, wherein the inner outside face includes a second lip section that is at least partially spaced-apart from a portion of the outer inside face.
14. The stethoscope chestpiece of any of embodiments 10 to 13, wherein part of the outer wall portion is coupled to the inner wall portion via a bridging element.
15. The stethoscope chestpiece of embodiment 14, wherein the bridging element is a bore tube.
16. The stethoscope chestpiece of embodiment 14, wherein the inner wall portion comprises a column and the column forms a portion of the bridging element.
17. The stethoscope chestpiece of embodiment 14, wherein the bridging element is disposed proximate to the outer lower edge perimeter and couples the outer lower edge with the inner lower edge.
18. The stethoscope chestpiece of any of embodiments 10 to 17, wherein part of the outer wall portion and part of the inner wall portion are integrally formed.
19. The stethoscope chestpiece of any of embodiments 10 to 18, wherein the outer wall portion and the inner wall portion are separate components.
20. The stethoscope chestpiece of embodiment 19, wherein the outer wall portion is releasably attached to the inner wall portion.
21. The stethoscope chestpiece of any of embodiments 10 to 20, wherein the stem fitting comprises a bore tube having a wall with an outer face and an inner face that forms a portion of a bore between the outer outside face and the second cavity therein, at least part of the bore tube is disposed in the first cavity.
22. The stethoscope chestpiece of any of embodiments 10 to 19, wherein the inner wall portion contacts the outer wall portion proximate to an apex.
23. The stethoscope chestpiece of any of embodiments 11 to 22, wherein the immobilizing element comprises an immobilizing element perimeter defining a contact between the immobilizing element and a diaphragm when the stethoscope chestpiece has pressure applied in a downward direction toward a patient.
24. The stethoscope chestpiece of embodiment 23, wherein the immobilizing element comprises a raised portion relative to a surface of the inner wall portion forming the second cavity.
25. The stethoscope chestpiece of embodiment 23 or 24, wherein a plane defined by the immobilizing element is above a plane defined by the outer lower edge toward the distal end.
26. The stethoscope chestpiece of any of embodiments 23 to 25, wherein the immobilizing element comprises an elastomeric ring.
27. The stethoscope chestpiece of any of embodiments 23 to 26, wherein the inner lower portion perimeter affects a frequency change of a diaphragm when downward pressure is applied.
28. The stethoscope chestpiece of any of embodiments 23 to 27, wherein the inner lower portion perimeter is the same as the immobilizing element perimeter.
29. An auscultation device comprising:

the stethoscope chestpiece of any of embodiments 1 to 9 or embodiments 10 to 28;

a diaphragm.
30. The auscultation device of embodiment 29, wherein the diaphragm is a single piece diaphragm.
31. The auscultation device of embodiment 29 and 30, further comprising

tubing;

a yoke having ear tips, the tubing and ear tips are fluidically coupled to the second cavity of the stethoscope chestpiece.
32. The auscultation device of any of embodiments 29 to 31, further comprising a microphone, a processor, and memory, the processor is configured to receive sounds from the microphone.
33. The auscultation device of any of embodiments 29 to 32, wherein a portion of the diaphragm defines a boundary of the first major cavity when a point an inner side of the diaphragm contacts the outer lower edge but not the immobilizing element, and a portion of the diaphragm defines a boundary of the second major cavity when at least a second point of the inner side of the diaphragm contacts the immobilizing element.
34. The auscultation device of any of embodiments 29 to 33, wherein the diaphragm defines a plane of the chestpiece which defines the first major cavity when a point an inner side of the diaphragm contacts the outer lower edge but not the immobilizing element.
35. A kit comprising:

the stethoscope chestpiece of any of embodiments 1 to 9 or embodiments 10 to 28;

a diaphragm.
36. A system comprising:

the auscultation device of any of embodiments 29 to 34:

a patient.
37. A method comprising:

providing a stethoscope having a chestpiece of any of embodiments 1 to 9 or embodiments 10 to 28;

contacting the patient; and

applying downward pressure to the stethoscope in a direction toward a patient.
38. The method of embodiment 37, wherein the pressure is capable of causing the diaphragm to contact the inner lower portion; and receiving sound at a 500 Hz frequency that differs by at least 5 decibels compared to a second stethoscope chestpiece having a ratio of a first major cavity to a second major cavity of less than 2.
38a. The method of embodiment 38, wherein the second stethoscope chestpiece is a Master Cardiology chestpiece by 3M.
39. The method of embodiment 38, comprising receiving sound at a 1000 Hz frequency that differs by at least 10 decibels compared to a second stethoscope chestpiece having a ratio of a first major cavity to a second major cavity of less than 2.
40. The method of embodiment 38, wherein the pressure is no greater than 100 g of force.
41. The method of embodiment 27, wherein the pressure is at least 1400 g of force; further comprising:

receiving sound at a 50 Hz frequency that differs by no greater than 5 decibels compared to a second stethoscope chestpiece having a ratio of a first major cavity to a second major cavity of less than 2.

In this application:

All headings provided herein are for the convenience of the reader and should not be used to limit the meaning of any text that follows the heading, unless so specified.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

Terms such as “a”, “an” and “the” are not intended to refer to only a singular entity, but include the general class of which a specific example may be used for illustration. The terms “a”, “an”, and “the” are used interchangeably with the term “at least one”.

The phrase “comprises at least one of” followed by a list refers to comprising any one of the items in the list and any combination of two or more items in the list. The phrase “at least one of” followed by a list refers to any one of the items in the list or any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

All numerical ranges are inclusive of their endpoints and nonintegral values between the endpoints unless otherwise stated (e.g., 1 to 5 includes 1, 1.5, 2, 2.75, 3, 3.80, 4, 5, etc.).

1. A stethoscope chestpiece, comprising:
a first major cavity formed from a portion of an outer wall portion, the outer wall portion comprises:
an outer lower edge forming an outer lower edge perimeter that establishes a bottommost plane;
a second major cavity formed from a portion of an inner wall portion having an immobilizing element disposed thereon, the second major cavity is within the first major cavity, a portion of the immobilizing element is spaced-apart with a portion of the outer lower edge perimeter;
wherein the first major cavity volume is at least two times a second major cavity volume.

2. The stethoscope chestpiece of claim 1, wherein the immobilizing element is adapted to be contacted by a diaphragm and to substantially immobilize the diaphragm when the diaphragm contacts the immobilizing element at a plane different than the bottommost plane so that the stethoscope chestpiece will pass low frequency sounds and attenuate high frequency sounds when the diaphragm is not contacting the immobilizing element, and, when the diaphragm is contacting the diaphragm, the acoustical stiffness of the diaphragm will be sufficiently higher than the first acoustical stiffness so that the head will pass high frequency sounds and attenuate low frequency sounds.

3. The stethoscope chestpiece of claim 1, further comprising: a bore tube positioned in the first major cavity, wherein a portion of the bore tube extends through the outer wall portion and is fluidically coupled with the first major cavity.

4. The stethoscope chestpiece of claim 1, wherein the outer wall portion comprises a distal end and a handle is formed from the outer wall portion proximate to the distal end.

5. The stethoscope chestpiece of claim 1, wherein the stethoscope chestpiece is single-sided.

6. The stethoscope chestpiece of claim 1, wherein the first major cavity volume is defined by empty space between the outer wall portion and the inner wall portion.

7. The stethoscope chestpiece of claim 1, wherein the immobilizing element is raised relative to a surface of the second major cavity.

8. The stethoscope chestpiece of claim 1, wherein the immobilizing element is an el astomeric ring.

9. The stethoscope chestpiece of claim 1, wherein, when a 500 to 1500 Hz frequency is received, a frequency response is at least 10 decibels below that of a second stethoscope chestpiece having a ratio of the first major cavity to the second major cavity of less than two.

10. A stethoscope chestpiece, comprising:
an outer wall portion having an outer inside face, an outer outside face, an outer lower edge, the outer lower edge forms an outer lower edge perimeter and defines a first plane;
an inner wall portion contained within the outer portion having an inner inside face, an inner outside face, an inner lower portion, the inner lower portion forms an inner lower portion perimeter and defined by a second plane that is positioned above the first plane towards a distal end, a portion of the inner lower portion perimeter is spaced-apart from a portion of the outer lower edge perimeter and a portion of the inner outside face is spaced-apart from the outer inside face;
wherein the outer wall portion forms a first cavity having a first volume and the inner wall portion forms a second cavity having a second volume, the first volume is at least 3 times the second volume; and
a stem fitting partially formed in the outer wall portion such that a portion of the outer outside face is fluidically coupled to the second cavity.

11. The stethoscope chestpiece of claim 10, further comprising an immobilizing element disposed proximate to the inner lower portion perimeter.

12. The stethoscope chestpiece of claim 10, wherein the outer outside face includes a first lip section for attaching a diaphragm thereto.

13. The stethoscope chestpiece of claim 12, wherein the inner outside face includes a second lip section that is at least partially spaced-apart from a portion of the outer inside face.

14. The stethoscope chestpiece of claim 1, wherein part of the outer wall portion is coupled to the inner wall portion via a bridging element.

15. The stethoscope chestpiece of claim 14, wherein the bridging element is a bore tube.

16. The stethoscope chestpiece of claim 14, wherein the inner wall portion comprises a column and the column forms a portion of the bridging element.

17. The stethoscope chestpiece of claim 14, wherein the bridging element is disposed proximate to the outer lower edge perimeter and couples the outer lower edge with the inner lower edge.

18. The stethoscope chestpiece of claim 1, wherein the immobilizing element comprises an immobilizing element perimeter defining a contact between the immobilizing element and a diaphragm when the stethoscope chestpiece has pressure applied in a downward direction toward a patient.

19. The stethoscope chestpiece of claim 18, wherein the immobilizing element comprises a raised portion relative to a surface of the inner wall portion forming the second cavity.

20. An auscultation device comprising:
the stethoscope chestpiece of claim 1; and
a diaphragm.
